(19) United States
(12) Patent Application Publication
(10) Pub. No.: US 2026/0092342 A1
(43) Pub. Date: Apr. 2, 2026

(54) ANTIBACTERIAL ALLOY MATERIAL FOR BIOMEDICAL IMPLANTS AND INSTRUMENTS

(71) Applicant: Titan64, Inc., Newark, DE (US)

(72) Inventors: Brandon Fields, Santa Clara, CA (US); Elliot Yedidiah Makhani, Los Angeles, CA (US)

(21) Appl. No.: 19/339,178

(22) Filed: Sep. 24, 2025

Related U.S. Application Data
(60) Provisional application No. 63/702,550, filed on Oct. 2, 2024.

(51) Int. Cl. C22C 14/00; A61K 6/84; A61L 31/02

(52) U.S. Cl. CPC C22C 14/00; A61K 6/84; A61L 31/022

(57) ABSTRACT

An antibacterial alloy material suitable for making biomedical implants and surgical instruments and devices associated with biomedical implantation is disclosed. The alloy material comprises about 50–99.99 wt.% Ti, about 1–49 wt.% Cu, and about 0.01–10 wt.% SiO₂. The antibacterial alloy material imparts antibacterial properties by enabling controlled ion release to inhibit biofilm formation, addressing issues like peri-implantitis associated with conventional bio-inert materials. In addition, the antibacterial alloy material enhances osseointegration and improves the mechanical durability of the resulting implants and surgical instruments and devices, including hardness and wear resistance. In addition, the antibacterial alloy material exhibits anti-corrosive properties and may be fabricated via techniques like spark plasma sintering (SPS).

### CROSS-REFERENCE TO RELATED APPLICATIONS

This application claims the benefit of U.S. Provisional Application No. 63/702,550, filed October 2, 2024, which is incorporated herein by reference in its entirety.

### TECHNICAL FIELD

This invention relates generally to alloy materials and, more particularly, to antibacterial alloy materials usable for making biomedical implants.

### BACKGROUND

The dental implant was a revolutionary invention that has changed the course of modern dentistry since its conception in 1965 and popularization in the following years. Prior to its advent, dentists had no way to permanently replace missing teeth in a way that completely mimicked natural dentition. The dental implant functions in an elegant way to mimic natural human anatomy. The implant body itself is a machined titanium rod ranging from 3-5 mm in diameter, and 7-16 mm in length. The rod is machined in such a way to have grooves that engage with cortical bone when placed by a dentist or oral surgeon. In this way, the implant body mimics the roots of a tooth, and a so-called “abutment” can be fabricated to mimic the crown of a tooth.

Despite this elegant design, the dental implant presents a unique challenge in maintenance due to its exposure to the oral cavity. The inherent challenge lies in the fact that the implant on which the prosthetic crown and abutment sit must be exposed to the oral cavity, which is an environment rich with bacterial flora. The abutment must interface with the implant body, and, as a result, the implant body itself will be exposed to this bacterial environment, making it susceptible to forming a biofilm of oral flora that may produce irritants and inflammatory factors that may irritate and damage both hard and soft tissue surrounding the implant.

The destructive hard and soft tissue irritation pathologies that may be caused by biofilm accumulation on dental implants have been named peri-implant-mucositis and peri-implantitis. Peri implant mucositis refers specifically to soft tissue inflammation and irritation surrounding a dental implant. It is analogous to gingivitis of a natural tooth and can be identified by bleeding upon probing with a dental probe, redness of the gingiva surrounding an implant, and other signs of swelling or inflammation. Peri-implant-mucositis can regress and reverse when the offending stimulus is removed. If peri-implant-mucositis is present, it can possibly be resolved following a proper dental cleaning to remove accumulated biofilm, and by improved oral hygiene at home.

Peri-implantitis is defined specifically as the loss of bone around an implant, and is analogous to periodontitis of natural teeth. It is generally agreed that bone loss of greater than 2 mm surrounding an implant that has been placed within a year is considered peri-implantitis. Peri-implantitis is irreversible, and often a surgical procedure must be performed to prevent further bone loss and loss of the implant. Current evidence suggests that poor plaque control is a major risk factor for developing peri-implantitis due to uncontrolled biofilm accumulation. Bacteria within the biofilm such as Aggregatibacter actinomycetemcomitans and Prevotella intermedia elicit an inflammatory response from surrounding native tissue in the form of leukotrienes and other targeting molecules. This response results in the degradation of bone surrounding the implant, resulting in peri-implantitis. Smoking and diabetes are also hypothesized to be risk factors in the development of peri-implantitis as they are major risk factors in developing periodontitis. However, data is still inconclusive to confirm these hypotheses.

To date, titanium has been the gold standard for dental implant material as it is biologically inert and is rarely rejected by the human body itself. However, it is not immune to biofilm accumulation and in fact is quite susceptible to forming such a biofilm in an environment such as the oral cavity.

### SUMMARY

Generally speaking, the present application is directed to antibacterial alloy materials suitable for making biomedical implants that are resistant to biofilm formation.

In some embodiments, a dental implant body comprises an alloy material, and the alloy material comprises about 50–99.99 wt.% Ti, about 1–49 wt.% Cu, and about 0.01–10 wt.% SiO₂.

In some embodiments, the alloy material of the dental implant body further comprises at least one of: about 0.01-10 wt.% ZrO_{2,} MgO, about 0.01-10 wt.% CaO, about 0.01-10 wt.% Ta₂O₅, about 0.01-10 wt.% Al₂O₃, about 0.01-47 wt.% Ag, about 0.01-50 wt.% Mg, and about 0.01-50 wt.% Zn.

In some embodiments, the alloy material of the dental implant body further comprises about 1–49 vol.% of a Ti₂Cu phase. In some embodiments, the alloy material of the dental implant body further comprises one of: about 0.01-47 vol.% of a Ti₂Ag phase, about 0.01-50 vol.% of a Ti₂Mg phase or Ti-Mg Intermetallic, and about 0.01-50 vol.% of a Ti₂Zn phase.

As used herein, the term “phase” refers to a specific atomic structure between any given elements such as Titanium, Copper, Silver, etc. More specifically, as used herein, the term “phase” refers to an intermetallic compound made from two or more metallic elements in a fixed chemical ratio, forming a distinct crystal structure and possessing properties that differ from its constituent elements and alloys. For example, Ti₂Cu is an example of a phase with a crystal structure and properties that are distinct from the properties of Ti and Cu separately.

In the context of the embodiments of alloys described herein, the Ti₂Cu phase exhibits antibacterial properties that are enhanced when compared to the antibacterial properties of Cu when used as a distinct and separate element of an alloy. As an example, the phases of Ti and Cu may include alpha-Titanium which is a hexagonal close packed structure and occurs at low copper content and beta-Titanium, which is a body centered cubic structure with higher copper content achieved by heat treatment.

Generally speaking, the Ti₂Cu phase is an intermetallic, typically tetragonal structure (which could be achieved by a heat treatment), and TiCu which is another intermetallic structure which could be cubic or tetragonal. Without wishing to be limited to theory such phase may form in an alloy as a result of relative percentages of each metal present in the alloy, the heat treatment(s) to which the alloy is subjected, and by thermodynamic favorability.

Without wishing to be limited by theory, the phase of interest of an alloy, for example, a Ti₂Cu phase, Ti₂Ag phase, Ti₂Mg phase, Ti₂Zn phase, etc., may be fine-tuned by applying a heat treatment at a temperature and for an amount of time aimed at achieving the desired weight percentage of the phase in the resulting alloy. Without wishing to be limited by theory, in some embodiments, an alloy comprising Ti, Cu, and SiO₂ may be made using a heat treatment and processing conditions that result in an alloy comprising about 1–49 vol.% of a Ti₂Cu phase. In some embodiments, an alloy material may be made using a heat treatment and processing conditions that result in an alloy comprising, for example, about 0.01-47 vol.% Ti₂Ag phase and/or about 0.01-50 vol.% Ti₂Mg phase and/or and about 0.01-50 vol.% Ti₂Zn phase.

For example, the Ti₂Cu phase in a Titanium-Copper-Silicon Dioxide alloy may be controlled by heating the alloy to a temperature of 790ºC to 1005ºC for a time of 1 hour to 10 hours, and then using SEM (scanning electron microscopy) and XRD (x-ray diffraction) to confirm the desired Ti₂Cu phase percentage is achieved in the alloy. Generally, due to its distinct atomic structure, the Ti₂Cu phase may possess its own unique advantageous properties such antibacterial effectiveness, osseointegration, mechanical properties, and/or anti-corrosion properties.

In some embodiments, a dental abutment comprises an alloy material, the alloy material comprising 50–99.99 wt.% Ti, about 1–49 wt.% Cu, and about 0.01–10 wt.% SiO₂.

In some embodiments, the alloy material of the dental abutment further comprises at least one of: about 0.01-10 wt.% ZrO_{2,} MgO, about 0.01-10 wt.% CaO, about 0.01-10 wt.% Ta₂O₅, about 0.01-10 wt.% Al₂O₃, about 0.01-47 wt.% Ag, about 0.01-50 wt.% Mg, and about 0.01-50 wt.% Zn.

In some embodiments, the alloy material of the dental abutment further comprises about 1–49 vol.% of a Ti₂Cu phase. In some embodiments, the alloy material of the dental abutment further comprises one of: 0.01-47 vol.% of a Ti₂Ag phase, about 0.01-50 vol.% of a Ti₂Mg phase or Ti-Mg Intermetallic, and about .01-50 vol.% of a Ti₂Zn phase.

In some embodiments, a surgical instrument comprises an alloy material, the alloy material comprising about 50–99.99 wt.% Ti, about 1–49 wt.% Cu, and about 0.01–10 wt.% SiO₂.

In some embodiments, the surgical instrument is one of: a surgical guide; an implant driver; a saw blade; a screwdriver comprising one or more bits; a surgical handpiece comprising one or more burs; a cover screw and healing abutment; a depth probe and guiding pin; and all surgical and dental armamentarium required for any biological implant placement or removal.

In some embodiments, the alloy material of the surgical instrument further comprises at least one of: about 0.01-10 wt.% ZrO_{2,} MgO, about 0.01-10 wt.% CaO, about 0.01-10 wt.% Ta₂O₅, about 0.01-10 wt.% Al₂O₃, about 0.01-47 wt.% Ag, about 0.01-50 wt.% Mg, and about 0.01-50 wt.% Zn.

In some embodiments, the alloy material of the surgical instrument further comprises about 1–49 vol.% of a Ti₂Cu phase. In some embodiments, the alloy material of the surgical instrument further comprises one of: 0.01-47 vol.% of a Ti₂Ag phase, about 0.01-50 vol.% of a Ti₂Mg phase or Ti-Mg Intermetallic, and about 0.01-50 vol.% of a Ti₂Zn phase.

In some embodiments, an orthopedic implant comprises an alloy material, the alloy material comprising about 50–99.99 wt.% Ti, about 1–49 wt.% Cu, and about 0.01–10 wt.% SiO₂.

In some embodiments, the orthopedic implant is one of: a jaw implant; a spinal implant; a hip implant; a shoulder implant; a fixation plate comprising a fixation screw; a reconstruction plate comprising a reconstruction screw; a pin; a wire; and a staple.

In some embodiments, the alloy material of the orthopedic implant further comprises at least one of: about 0.01-10 wt.% ZrO_{2,} MgO, about 0.01-10 wt.% CaO, about 0.01-10 wt.% Ta₂O₅, about 0.01-10 wt.% Al₂O₃, about 0.01-47 wt.% Ag, about 0.01-50 wt.% Mg, and about 0.01-50 wt.% Zn.

In some embodiments, the alloy material of the orthopedic implant further comprises about 1–49 vol.% of a Ti₂Cu phase. In some embodiments, the alloy material of the orthopedic implant further comprises one of: 0.01-47 vol.% of a Ti₂Ag phase, about 0.01-50 vol.% of a Ti₂Mg phase or Ti-Mg Intermetallic, and about 0.01-50 vol.% of a Ti₂Zn phase.

### BRIEF DESCRIPTION OF DRAWINGS

Disclosed herein are embodiments of antibacterial alloy compositions and biomedical implants that may be made from such antibacterial compositions. This description includes drawings, wherein:

FIG. 1 shows an example dental implant body utilizing the antibacterial alloy material in accordance with some embodiments;

FIG. 2 shows an example dental abutment and screw utilizing the antibacterial alloy material in accordance with some embodiments;

FIG. 3 shows example dental surgical guides utilizing the antibacterial alloy material in accordance with some embodiments;

FIG. 4A shows an example dental implant driver utilizing the antibacterial alloy material in accordance with some embodiments;

FIG. 4B shows an example bit utilizing the antibacterial alloy material in accordance with some embodiments and associated with the example dental implant driver shown in FIG. 4A;

FIG. 5 shows an example surgical bone saw blade utilizing the antibacterial alloy material in accordance with some embodiments;

FIG. 6 shows an example orthopedic implant utilizing the antibacterial alloy material in accordance with some embodiments;

FIG. 7 shows example bone fixation plates utilizing the antibacterial alloy material in accordance with some embodiments;

FIG. 8 shows an example bone fixation plate and associated fixation screws utilizing the antibacterial alloy material in accordance with some embodiments;

FIG. 9 shows an example spinal disc replacement implant utilizing the antibacterial alloy material in accordance with some embodiments;

FIG. 10 shows a graph indicating antibacterial properties of several antibacterial alloy materials in accordance with some embodiments;

FIG. 11 shows a graph indicating corrosion testing of several antibacterial alloy materials in accordance with some embodiments;

FIGS. 12A-12D show scanning electron micrograph (SEM) images of several embodiments of the alloy and illustrate the overall composition, the intermetallic compound (Ti₂-Cu phase) percentage, and microstructure (phase) present in the illustrated alloy embodiments.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments. Also, common but well-understood elements that are useful or necessary in a commercially feasible embodiment are often not depicted in order to facilitate a less obstructed view of these various embodiments. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein.

### DETAILED DESCRIPTION

The following description is not to be taken in a limiting sense, but is made merely for the purpose of describing the general principles of example embodiments. Reference throughout this specification to “one embodiment,” “an embodiment,” “some embodiments,” “an implementation,” “some implementations,” “some applications,” or similar language means that a particular feature, structure, or characteristic described in connection with the embodiment is included in at least one embodiment of the invention. Thus, appearances of phrases “in one embodiment,” “in an embodiment,” “in some embodiments,” “in some implementations,” and similar language throughout the specification may, but do not necessarily, all refer to the same embodiment.

Titanium and various titanium (Ti) alloys (e.g., 90% Ti, 6% Aluminum (Al) and 4% Vanadium (V)) are commonly used to make orthopedic implants, primarily due to their biocompatibility and osseointegrative effect. Generally, Ti alloys have allowed for vast improvements compared to previous generations of orthopedic implants consisting of stainless steels, cobalt-chrome, and many other metallic alloys. For example, Ti is not only more biocompatible compared to previous materials, but also compatible with x-ray computed tomography and magnetic resonance imaging thanks to its magnetic structure and properties.

However, bio-inert Ti alloys do not solve a major pitfall associated with conventional implant design. In particular, while bio-inert Ti implants reduce the chance of rejection by the immune system of the subject, they also allow bacteria to grow uninhibited on the implant. To address this, different antibacterial solutions have been previously proposed. For example, hydroxyapatite (HA) is a common coating used in dental implants which, unlike pure titanium alloy stimulates osseointegration. It is known to add antibacterial agent to HA coatings to prevent growth of common bacterial strains. However HA coatings are known to be unstable and are susceptible to wear and flaking over time.

Copper is a well-known antibacterial agent. The long-term safety and efficacy of copper have been well studied through approximately 150 million cases of implementation in intrauterine devices worldwide. While excessive/extreme levels of copper, just like excessive concentrations of other metals, may exhibit toxicity, if the metal (i.e., copper) ion release rate is below certain safe threshold amounts in the body, then that device can be used safely. One way to introduce copper into the body apart from its pure elemental form (as is done in an IUD device) is by way of a copper-containing alloy material. Titanium copper alloys with as little as 3 wt.% copper have shown to exhibit antibacterial properties in in vitro bacterial tests, albeit with a lower osseointegration when compared to pure Ti.

This application describes embodiments of an antibacterial alloy material that may be advantageously used to make various implantable devices (also referred to herein generally as “implants”), including but not limited to: dental implant body, dental abutment and associated screw(s), dental (and non-dental) surgical guides, dental and non-dental implant drivers, bits associated with surgical guides, surgical bone saw blades, orthopedic implants, bone (e.g., jaw) fixation plates, fixation screws, and spinal disc replacement implants. Some example devices that may be implanted into a human or animal subject and that may be made of the antibacterial alloys described herein are depicted in the drawing figures and discussed below.

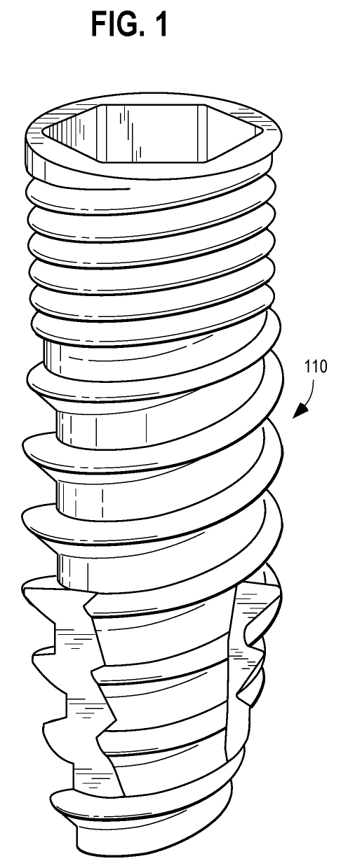

FIG. 1 illustrates an example dental implant body 110 made of the antibacterial alloy material in accordance with some embodiments described herein. Because the dental implant body 110 is made of the antibacterial alloy according to the embodiments described herein, the dental implant body 110 will exhibit superior antibacterial, osseointegrative, and anti-corrosive properties when compared to a dental implant body made of a conventional material (e.g., Ti) or a conventional alloy (e.g., a dental implant body made of 90 wt. % Ti, 6 wt.% Al, and 4 wt.% V). It will be appreciated that the shape and size of the dental implant body 110 has been shown in FIG. 1 by way of example only, and that the antibacterial alloy materials according to the embodiments described herein may be used to fabricate a dental implant body of any suitable size and shape.

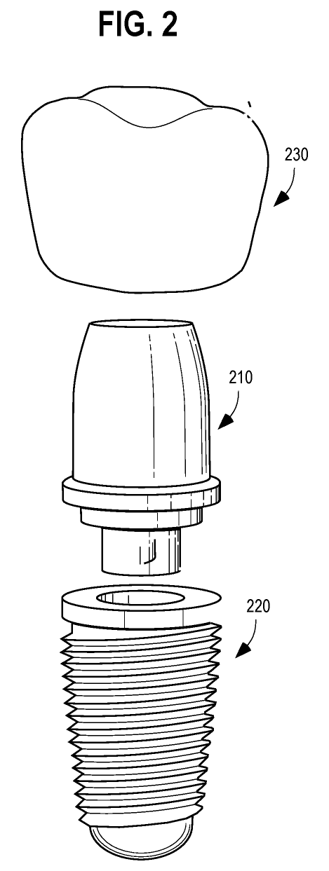

FIG. 2 shows an example assembly including a dental abutment 210 and associated screw 220 that, in combination, facilitate the installation of a dental crown 230. Because the dental abutment 210 and screw 220 are each made of the antibacterial alloy according to the embodiments described herein, the dental abutment 210 and screw 220 will exhibit superior antibacterial, osseointegrative, and anti-corrosive properties when compared to dental abutments and associated screws that are made of conventional materials and/or alloys. It will be appreciated that the shape and size of the dental abutment 210 and screw 220 has been shown in FIG. 2 by way of example only, and that the antibacterial alloy materials according to the embodiments described herein may be used to fabricate dental abutments 210 and screws 220 of any suitable size and shape.

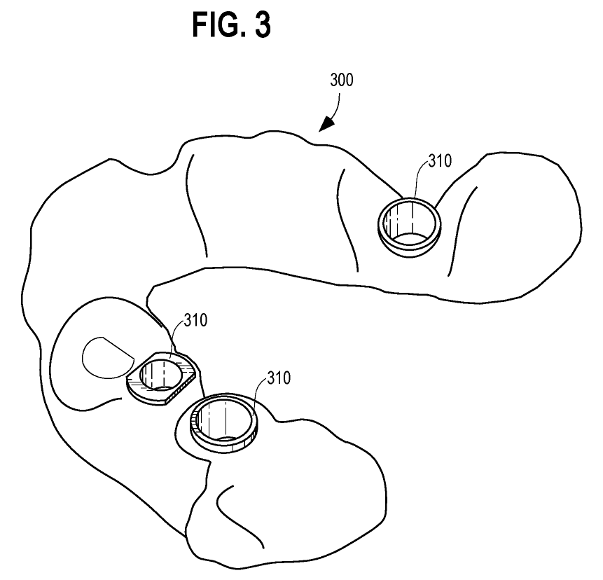

FIG. 3 shows an example dental surgical guide 300 that includes drill Sleeves (also referred to as guiding cylinders) 310 that are made of the antibacterial alloy material in accordance with some embodiment. Because the drill sleeves 310 are made of the antibacterial alloy according to the embodiments described herein, the drill sleeves 310 will exhibit superior antibacterial and anti-corrosive properties when compared to drill sleeves made of conventional materials and conventional alloys. It will be appreciated that the shape and size of the dental surgical guide 300 and the drill sleeves 310 have been shown in FIG. 3 by way of example only, and that the antibacterial alloy materials according to the embodiments described herein may be used to fabricate drill sleeves 310 of any suitable size and shape.

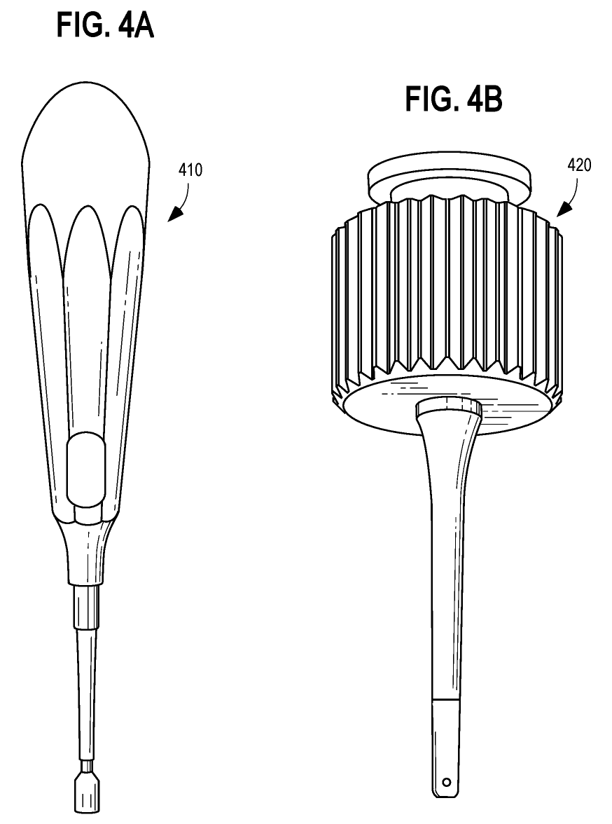

FIG. 4A and FIG. 4B illustrate examples of surgical tools and instruments associated with implantation procedures, namely, a dental implant driver 410 (FIG. 4A) and a bit 420 (FIG. 4B) that may be used in conjunction with the dental implant driver 410. Because the dental implant driver 410 and bit 420 are made of the antibacterial alloy according to the embodiments described herein, the dental implant driver 410 and bit 420 will exhibit superior antibacterial properties and anti-corrosive properties when compared to implant drivers and bits made of conventional materials and conventional alloys. It will be appreciated that the shape and size of the dental implant driver 410 and bit 420 have been shown in FIGS. 4A and 4B by way of example only, and that the antibacterial alloy materials according to the embodiments described herein may be used to fabricate dental implant drivers 410 and bits 420 (and non-dental implant drivers and associated bits, as well as other surgical implants used in conjunction with implantation) of any suitable size and shape.

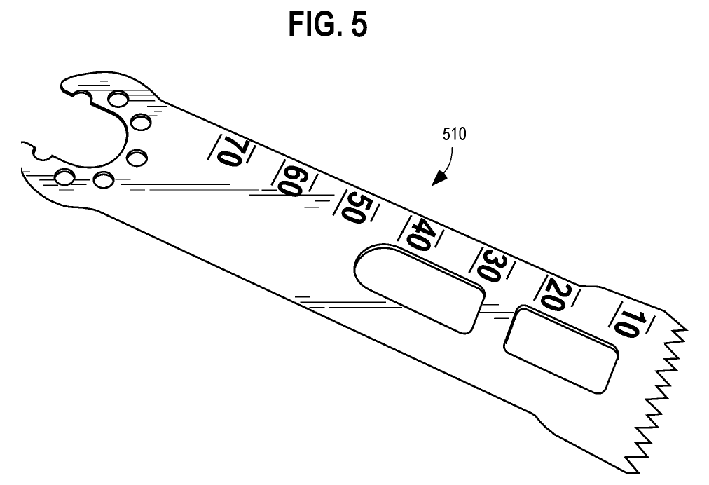

FIG. 5 shows an example surgical bone saw blade 510 made of the antibacterial alloy according to the embodiments described herein. Because the surgical bone saw blade 510 is made of the antibacterial alloy according to the embodiments described herein, the surgical bone saw blade 510 will exhibit superior antibacterial properties and anti-corrosive properties when compared to surgical bone saw blades made of conventional materials and conventional alloys. It will be appreciated that the shape and size of the surgical bone saw blade 510 have been shown in FIG. 5 by way of example only, and that the antibacterial alloy materials according to the embodiments described herein may be used to fabricate surgical bone saw blades of any suitable type, size, and shape.

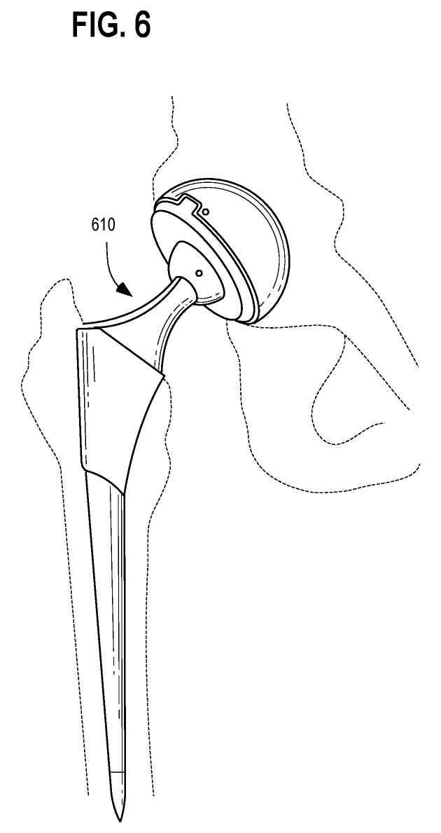

FIG. 6 illustrates an example orthopedic (in this example, a hip) implant 610 made of the antibacterial alloy material in accordance with some embodiments described herein. Because the orthopedic implant 610 is made of the antibacterial alloy according to the embodiments described herein, the orthopedic implant 610 will exhibit superior antibacterial, osseointegrative, and anti-corrosive properties when compared to an orthopedic implant made of a conventional material or a conventional alloy. It will be appreciated that the shape and size of the orthopedic implant 610 has been shown in FIG. 6 by way of example only, and that the antibacterial alloy materials according to the embodiments described herein may be used to fabricate hip implants (and other types of orthopedic implants) of any suitable size and shape.

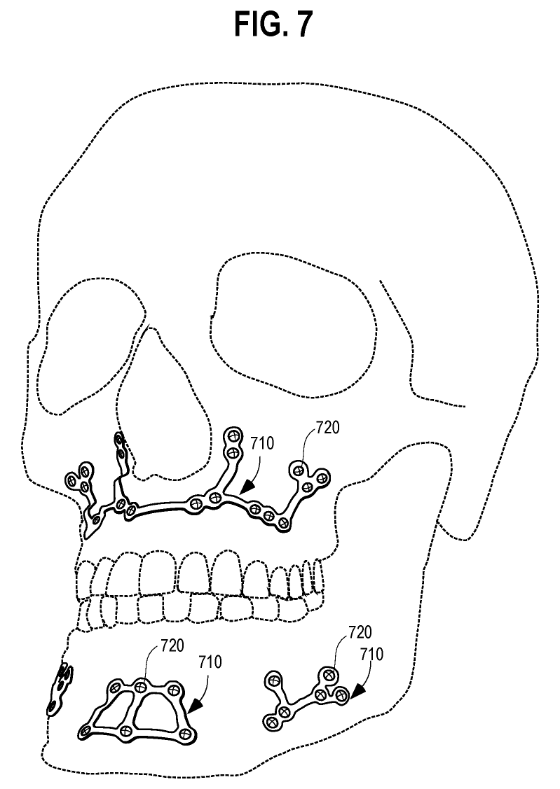

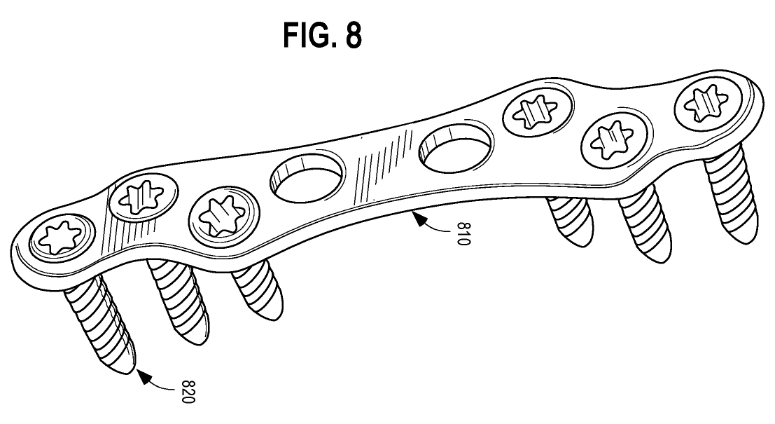

FIGS. 7 and 8 show examples bone fixation plates 710, 810 made of the antibacterial alloy material in accordance with some embodiments described herein, as well as screws 720, 820 made of the antibacterial alloy material in accordance with some embodiments described herein that may be used with the bone fixation plates 710, 810. Because the bone fixation plates 710, 810 and the screws 720, 820 are made of the antibacterial alloys according to the embodiments described herein, the bone fixation plates 710, 810 and the screws 720, 820 will exhibit superior antibacterial, osseointegrative, and anti-corrosive properties when compared to bone fixation plates and screws made of a conventional material or a conventional alloy. It will be appreciated that the shapes and sizes of the bone fixation plates 710, 810 and screws 720, 820 has been shown in FIGS. 7 and 8 by way of example only, and that the antibacterial alloy materials according to the embodiments described herein may be used to fabricate bone fixation plates and screws of any suitable size and shape.

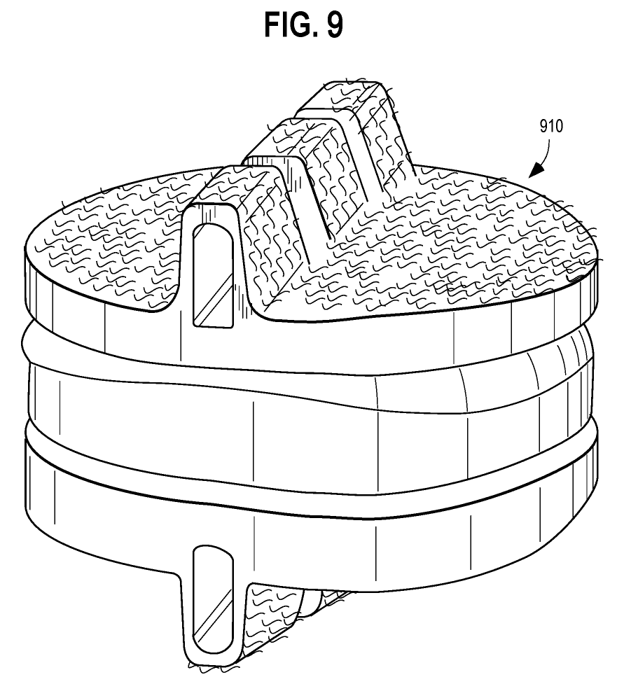

FIG. 9 illustrates an example orthopedic (in this example, an intervertebral spinal) implant 910 made of the antibacterial alloy material in accordance with some embodiments described herein. Because the orthopedic implant 910 is made of the antibacterial alloy according to the embodiments described herein, the orthopedic implant 910 will exhibit superior antibacterial, osseointegrative, and anti-corrosive properties when compared to an orthopedic (e.g., spinal) implant made of a conventional material or a conventional alloy. It will be appreciated that the shape and size of the orthopedic implant 910 has been shown in FIG. 9 by way of example only, and that the antibacterial alloy materials according to the embodiments described herein may be used to fabricate spinal (and other orthopedic) implants of any suitable size and shape.

In some embodiments the implants described herein may include up to 50 wt.% (e.g., from about 1 wt.% to about 50 wt.%) copper, alone, or combined with at least one of silver (Ag), magnesium (Mg) and zinc (Zn) as an antibacterial component. Without wishing to be limited by theory, having a precise amounts and/or combinations of the antibacterial elements in the alloy allows for formation of highly antibacterial ion-releasing phases, which are significantly less toxic than a pure copper phase.

As mentioned above, copper is well-established as effective as an antibacterial agent at low-ion release rates, which has been well-demonstrated as safe through extensive use in intrauterine devices. The antibacterial alloy embodiments described herein may include copper as the only antibacterial agent in an optimal weight percentage, but may also include copper in combination with one or both of silver and zing in optimal weight percentages and relative ratios. In some implementations, the antibacterial alloys may include silver and/or zinc when suitable to address an allergy and/or to improve differential efficacy. Without wishing to be limited by theory, inclusion of copper and or zinc and/or silver in embodiments of antibacterial alloys may enhance surface activity of the resulting implants and significantly reduce bacterial growth on such implants when compared to possible bacterial grown on traditional titanium only alloys.

Generally speaking, and as described in more detail below, certain embodiments of the implants made from the alloys described herein show a bacterial reduction of over 95% against Staphylococcus aureus and Escherichia coli within 24 hours of contact, as tested by agar diffusion and direct bacterial adhesion assays. Furthermore, embodiments of the implants described herein effectively inhibit biofilm formation, thereby mitigating the risk of implant-associated infections.

Notably, pure copper is known to soften the alloy and undesirably decrease osseointegration. The embodiments of the alloys described herein, unlike pure titanium and conventional Ti-6Al-4V alloys, advantageously include up to 10 wt.% (e.g., from about 0.01 wt. % to about 10 wt.%) silicon dioxide (SiO₂) alone, or in combination with at least one of zirconium oxide (ZrO₂), magnesium oxide (MgO), (calcium oxide) CaO, tantalum pentoxide (Ta₂O₅), and aluminum oxide (Al₂O₃) to facilitate osseointegration. Without wishing to be limited by theory, the inclusion of silicon dioxide, aluminum oxide, and/or zirconium oxide in the antibacterial alloys used to make the example implants described herein may facilitate the attachment of bone to the implants and stimulate osteoblast activity, thereby advantageously enhancing osseointegration of the implants. This feature of the example implants described herein overcomes a common limitation of implants made from pure titanium alloys, which, while biocompatible, are bio-inert, but allow uncontrolled growth of bacteria thereon.

In some embodiments described herein the implants comprise an antibacterial alloy material that includes only Ti, Cu, and SiO₂. In other embodiments, the implants comprise an antibacterial alloy material that includes Ti, Cu, and SiO₂ combined with at least one of Ag, Mg, Zn, ZrO₂, MgO, CaO, Ta₂O₅, Al₂O_{3.} Advantageously, the implants according to the embodiments described herein do not include (and do not need) any additional exterior osteoinductive coating (although, as mentioned below, another suitable coating may be added in some embodiments). Notably, since the implants according to the embodiments described herein may be made without an exterior coating, the manufacturing process of such implants is simpler when compared to the manufacturing process of implants that do require an exterior coating.

When made from the antibacterial alloys described herein, the implants advantageously possess both antibacterial properties and osseointegration. In some aspects, the implants may include a coating, for example a coating comprising 90 wt. % Ti, 5 wt.% Cu, and 5 w.% SiO₂.

In various embodiments, the antibacterial alloys and implants disclosed herein may be fabricated via spark plasma sintering (SPS). The precise control over the sintering process afforded by SPS enables the formation of a fine-grained, homogeneous microstructure. Such a controlled fabrication process yields materials with improved physical characteristics and enhanced performance for use in implantable devices. In addition, the fine distribution of copper, zinc, silver, silicon dioxide, aluminum oxide, magnesium oxide, calcium oxide, tantalum oxide, and/or zirconium oxide particles within the titanium matrix ensures that the release of Cu²⁺, Mg²⁺ Zn²⁺, and/or Ag⁺ ions is gradual and continuous, enhancing the antibacterial efficacy of the material over time while also making it non-toxic to the host.

In addition, this microstructural uniformity advantageously minimizes grain growth and enhances the mechanical properties of the resulting alloy, including but not limited to, hardness and tensile strength. For example, the tensile strength of the example implants described herein is in the range of 700-900 MPa, which is a strength that is suitable for load-bearing implants. In addition, the hardness of the example implants described herein may be up to about 350 HV. Without wishing to be limited by theory, the inclusion of silicon dioxide in the alloy materials used to make the implants enhances the hardness and improves the surface wear resistance of the implants, which is important for implants that experience frictional forces (e.g., joint implants).

Notably, the implants described herein do not necessarily have to be prepared using SPS and it will be appreciated that other suitable fabrication techniques may be used to manufacture implants according to the embodiments described herein. Such alternative fabrication techniques may include but are not limited to: sputtering physical vapor deposition, machining, surface etching, etc. For example, a sputtering physical vapor deposition process may be used to apply a coating of the antibacterial alloy material onto a commercially available implant. This can serve to enhance the implant's antibacterial, osseointegrative, and/or mechanical properties. Machining or surface etching may be used to modify the surface of an implant to enhance its mechanical, antibacterial, and/or osseointegrative properties, reduce corrosion, or simply to enhance its aesthetic qualities.

In certain embodiments, the surface of any of the implants described herein may be modified to provide enhanced functionality. One possible surface modification is anodization, which creates a controlled, porous oxide layer on the surface of the antibacterial alloy material of the implant. Without wishing to be limited by theory, this anodized surface may promote bone cell adhesion and facilitate the sustained release of antibacterial ions, such as copper (Cu₂+) ions. Alternative surface modifications may also be employed, including plasma spraying or the application of hydroxyapatite (HA) coatings, to further enhance osteoconductivity while maintaining the inherent advantageous antibacterial properties of the alloy material.

Surface Modifications for Enhanced Functionality: Preferred Surface Modification: Anodization to create a controlled porous oxide layer on the titanium surface, promoting both bone cell adhesion and sustained copper ion release. Alternative Modifications: Plasma spraying or hydroxyapatite coatings can be used to enhance osteoconductivity while maintaining the antibacterial functionality of the material.

When fabricating implants from the antibacterial alloys described herein, the resulting implants advantageously possess antibacterial properties and enhanced osseointegration, as well as long-term durability and biocompatibility, making such implants ideal for use in orthopedic, spinal, dental applications, as well as in any other body implant applicants that currently rely on pure titanium implants or titanium-aluminum-vanadium implants.

### EXAMPLES

Advantages and embodiments of the antibacterial alloys and their applications described herein are further illustrated by the following examples; however, the particular conditions, processing schemes, materials, and amounts thereof recited in these examples should not be construed to unduly limit the overall scope of the contemplated compositions.

Throughout this specification, all percentages recited herein are by weight unless specified otherwise. In addition, throughout this specification, the term “about,” when used to precede a numerical value of a weight percentage, will be understood to be equal to the indicated numerical value plus and/or minus 0.01.

### Example 1. Machined Dental Implant:

Machining stock rods comprising 88wt% Ti, 1wt% Cu, 5wt% Zn, 5wt% Al₂O₃, 1wt% SiO₂ (which are referred to herein in shorthand as “Ti-1Cu-5Zn-5Al₂O₃-1SiO₂”) were heat-treated at 900°C for 2 hours for homogenizing before being machined into a dental implant screw and abutment using a computer numerical control (CNC) machine (i.e., a type of an automated manufacturing equipment that used computer software to control the movement of tools).

### Example 2: Coated Dental Implants:

Elemental powders are mixed to form a sputtering target with the following composition: 80 wt% Ti, 10 wt% Cu, 7 wt% SiO2, and 3 wt% Al2O3. This target, referred to in shorthand as “Ti-10Cu-7SiO2-3Al2O3”, serves as the feedstock coating material. The sputtering target may serve serves as feedstock coating material that may be used dental implants, surgical plates, and surgical guides.

### Example 3. Machined Dental Implant:

Machining stock rods comprising 50wt% Ti, 35wt% Cu, 5wt% Zn, 5wt% Al₂O₃, 5wt% SiO₂ (which are referred to herein in shorthand as “Ti-35Cu-5Zn-5Al₂O₃-5SiO₂”) were heat-treated at 900°C for 2 hours for homogenizing before being machined into a dental implant screw and abutment using a computer numerical control (CNC) machine (i.e., a type of an automated manufacturing equipment that used computer software to control the movement of tools).

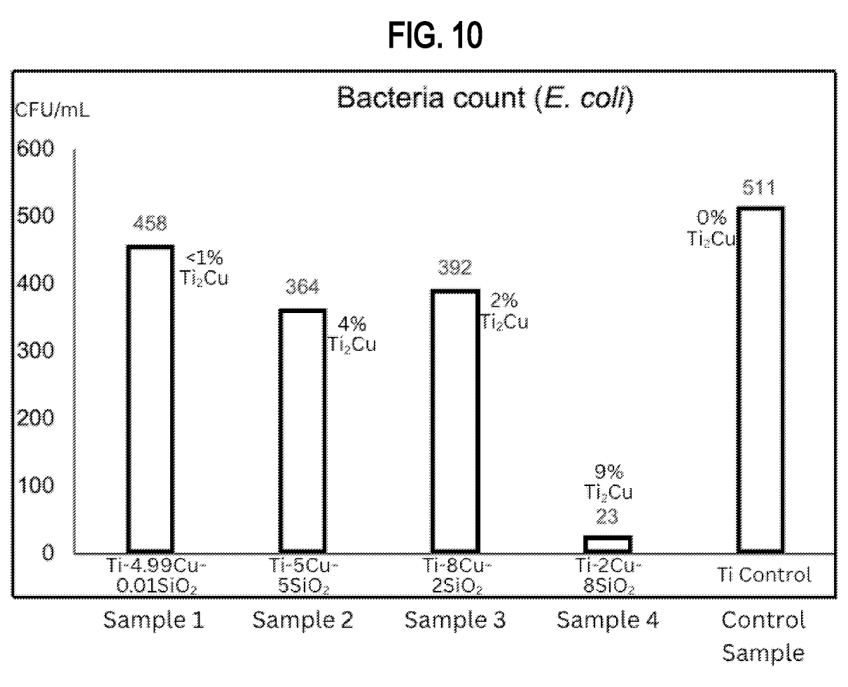

FIG. 10 shows the results of an experiment performed to test the antibacterial properties of the alloys according to some embodiments relative to a control (i.e., titanium only material). The antibacterial tests the results of which are shown in FIG. 10 were performed in a biosafety level 2 (BSL-2) lab. Escherichia coli (E. coli) bacteria were cultured overnight in media, and distributed on the surfaces of the tested alloys using inoculating loops, and then transferred to media in well plates. Metal pieces were transferred from well plates to Eppendorf tubes, then vortexed to distribute the bacteria. Serial dilution steps ensured a suitable number of cells for colony-counting after cultivation. 100 ul of the final dilution were transferred to each agar plate with precision micro-pipettes, then distributed with inoculating loops to make the coating uniform. Bacteria were incubated on agar plates for 24 hours at 37^{o}C before colony counting.

As be seen in FIG. 10, the antibacterial properties of five samples of materials were tested against E. coli, namely, Sample 1 (95 wt.% Ti and 5 wt.% Cu), Sample 2 (90 wt.% Ti, 5 wt.% Cu, and 5 wt.% SiO₂), Sample 3 (90 wt.% Ti, 8 wt.% Cu, and 2 wt.% SiO₂), Sample 4 (90 wt.% Ti, 2 wt.% Cu, and 8 wt.% SiO₂), and Control Sample (100 wt.% Ti). The X-axis in FIG. 10 identifies each of the five samples and indicates the composition of each of the five samples, and the Y-axis in FIG. 10 shows bacterial colony-forming units per milliliter (CFU/mL) measured on each sample.

FIG. 10 shows that the Control Sample (i.e., a material made of 100 wt.% titanium which had a 0% Ti₂Cu phase due to the absence of copper) had the highest bacterial growth thereon, which means that the material from which the Control Sample was made had the weakest antibacterial properties of the five samples tested. On the other hand, Sample 4, an alloy made of 90 wt.% titanium, 2 wt.% copper, and 8 wt.% silicon oxide with a Ti₂Cu phase of 9% had the lowest bacterial growth thereon, which means that the material from which Sample 4 was made had the strongest antibacterial properties of the five samples tested.

FIG. 10 further shows that Sample 2, an alloy made of 90 wt.% titanium, 5 wt.% copper, and 5 wt.% silicon oxide with a Ti₂Cu phase of 4% had the second lowest bacterial growth thereon, which means that the material from which Sample 2 was made had the second strongest antibacterial properties of the five samples tested. In addition, FIG. 10 shows that Sample 3, an alloy made of 90 wt.% titanium, 8 wt.% copper, and 2 wt.% silicon oxide with a Ti₂Cu phase of 2% had the third lowest bacterial growth thereon, which means that the material from which Sample 3 was made had the third strongest antibacterial properties of the five samples tested. Finally, FIG. 10 shows that Sample 1 (i.e., an alloy made of 95 wt.% titanium, 4.99 wt.% copper, and 0.01 wt.% silicon dioxide with a Ti₂Cu phase of less than 1% had the fourth lowest bacterial growth thereon, which means that the material from which Sample 1 was made had the fourth strongest antibacterial properties of the five samples tested.

As illustrated in FIG. 10, Sample 4 contains the lowest overall weight percentage of copper among Samples 1–4. Nevertheless, Sample 4 demonstrates the highest antibacterial activity of all the samples tested. Without being bound by theory, it is believed that this unexpectedly superior antibacterial performance is attributable to Sample 4 having the highest weight percentage of the Ti₂Cu intermetallic phase relative to Samples 1–3. Stated differently, FIG. 10 indicates that while the antibacterial performance of the Ti–Cu–SiO₂ alloys may be partially correlated with the overall copper content of the alloy, it is more strongly correlated with the amount of the Ti₂Cu phase present. Accordingly, the data suggest that the formation and concentration of the Ti₂Cu phase play a dominant role in determining the antibacterial efficacy of the alloy.

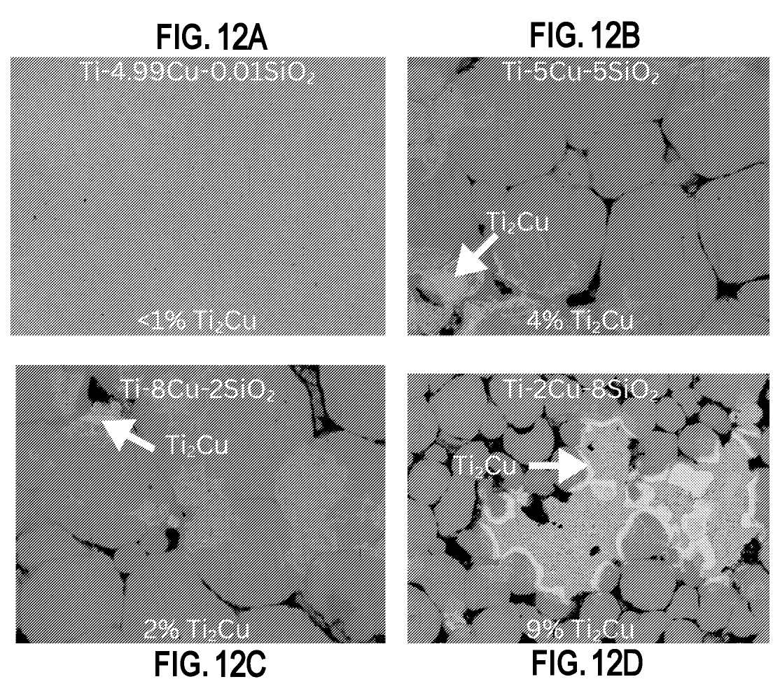

FIGS. 12A-12D show scanning electron micrograph (SEM) images of alloy Samples 1-4, and illustrate the composition of the alloy, the intermetallic compound (Ti₂Cu phase) percentage, and more detail regarding the differences in the microstructure of the illustrated alloy embodiments.

In particular, FIG. 12A shows a scanning electron micrograph (SEM) image of Sample 1, which is an alloy made of 95 wt.% titanium, 4.99 wt.% copper, and 0.01 wt.% silicon dioxide with a Ti₂Cu phase of less than 1%. FIG. 12B shows a scanning electron micrograph (SEM) image of Sample 2, an alloy made of 90 wt.% titanium, 5 wt.% copper, and 5 wt.% silicon oxide with a Ti₂Cu phase of 4%. FIG. 12C shows a scanning electron micrograph (SEM) image of Sample 3, an alloy made of 90 wt.% titanium, 8 wt.% copper, and 2 wt.% silicon oxide with a Ti₂Cu phase of 2%. FIG. 12D shows a scanning electron micrograph (SEM) image of Sample 4, an alloy made of 90 wt.% titanium, 2 wt.% copper, and 8 wt.% silicon oxide with a Ti₂Cu phase of 9%.

Collectively, FIGS. 12A-12D illustrate the differences in the microstructure between Samples 1-4, and illustrate that variations in the percentage of the intermetallic compound (Ti₂-Cu phase) present in the alloys result in visible variations in the microstructure of the alloys, which in turn affect at least the anti-bacterial properties of the alloys.

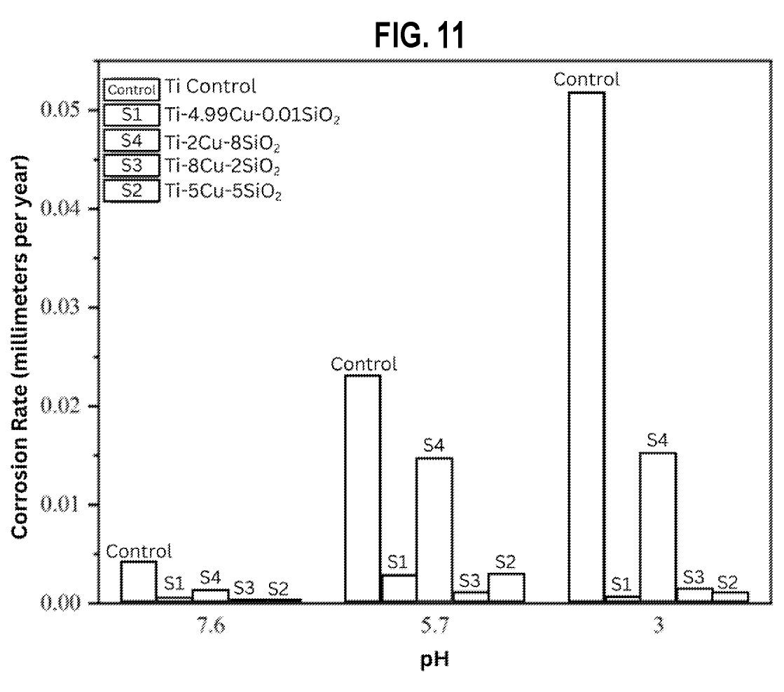

FIG. 11 shows the results of an experiment performed to test the anti-corrosion properties of the alloys according to some embodiments relative to a control alloy. In this experiment, electrochemical measurements were performed on polished samples at room temperature (24 ± 1°C) using a SP-300 Potentiostat (Bio-Logic). A conventional three-electrode cell was used, with an Ag/AgCl (3.0 M KCl) reference electrode and platinum sheet as a counter electrode. and the sample as working electrode. The electrochemical tests were performed in Carter Brugirard artificial saliva, prepared using deionized water and high purity chemicals purchased from Sigma-Aldrich. The chemicals were added to 100 mL of deionized water and stirred until dissolution, while the pH of the solution was constantly monitored using a pH meter (Accumet AE150 from Fisher Scientific). Solutions with three different pH values were prepared for this study; pH values of 7.6, 5.7 and 3.0. The pH was 7.6 immediately after preparing the solution using the compositions. More acidic pH values were obtained by adding lactic acid (88% - 92%) to the solution. The room temperature potentiodynamic polarization curves were measured with a scanning rate of 1 mV/s from -0.200V to 0.200V and -2V to 2V separately. The corrosion behavior of the samples was determined using Tafel analysis.

As be seen in FIG. 11, the corrosion rates of five samples of alloy materials were tested, Sample 1 (95 wt.% Ti and 5 wt.% Cu), Sample 2 (90 wt.% Ti, 2 wt.% Cu, and 8 wt.% SiO₂), Sample 3 (90 wt.% Ti, 8 wt.% Cu, and 2 wt.% SiO₂), Sample 4 (90 wt.% Ti, 5 wt.% Cu, and 5 wt.% SiO₂), and Control Sample (i.e., 90 wt.% Ti, 6 wt.% Al, and 4 wt.% V). In FIG. 11, the X-axis is pH and the Y-axis is corrosion rate (CR), which is represented in mmpy (millimeters per year). FIG. 11 shows that each of samples 1-4 had significantly less susceptible to corrosion in comparison to the Control Sample.

Those skilled in the art will recognize that a wide variety of other modifications, alterations, and combinations can also be made with respect to the above-described embodiments without departing from the scope of the invention, and that such modifications, alterations, and combinations are to be viewed as being within the ambit of the inventive concept.

1. A dental implant body comprising an alloy material, the alloy material comprising about 50–99.99 wt.% Ti, about 1–49 wt.% Cu, and about 0.01–10 wt.% SiO₂.

2. The dental implant body of claim 1, wherein the alloy material further comprises at least one of: about 0.01-10. wt.% ZrO_{2,} MgO, about 0.01-10. wt.% CaO, about 0.01-10. wt.% Ta₂O₅ about 0.0-01. wt.% Al₂O₃ about 0.01-10. wt.% Ag, about 0.01-10. wt.% Mg, and about 0.01-10. wt.% Zn.

3. The dental implant body of claim 1, wherein the alloy material further comprises about 1–49 vol.% of a Ti₂Cu phase.

4. The dental implant body of claim 2, wherein the alloy material further comprises one of: about 0.01-47. vol.% of a Ti₂Ag phase, about 0.01-50. vol.% of a Ti₂Mg phase or Ti-Mg Intermetallic, and about 0.01-50. vol.% of a Ti₂Zn phase.

5. A dental abutment comprising an alloy material, the alloy material comprising 50–99.99 wt.% Ti, about 1–49 wt.% Cu, and about 0.01–10 wt.% SiO₂.

6. The dental abutment of claim 5, wherein the alloy material further comprises at least one of: about 0.01-10. wt.% ZrO_{2,} MgO, about 0.01-10. wt.% CaO, about 0.01-10. wt.% Ta₂O₅ about 0.01-10. wt.% Al₂O₃ about 0.01-47. wt.% Ag, about 0.01-50. wt.% Mg, and about 0.01-50. wt.% Zn.

7. The dental abutment of claim 5, wherein the alloy material further comprises about 1–49 vol.% of a Ti₂Cu phase.

8. The dental abutment of claim 6, wherein the alloy material further comprises one of: about 0.01-47. vol.% of a Ti₂Ag phase, about 0.01-50. vol.% of a Ti₂Mg phase or Ti-Mg Intermetallic, and about .01-50. vol.% of a Ti₂Zn phase.

9. A surgical instrument comprising an alloy material, the alloy material comprising about 50–99.99 wt.% Ti, about 1–49 wt.% Cu, and about 0.01–10 wt.% SiO₂.

10. The surgical instrument of claim 9, wherein the surgical instrument is one of:
a surgical guide;
an implant driver;
a saw blade;
a screwdriver comprising one or more bits;
a surgical handpiece comprising one or more burs;
a cover screw and healing abutment;
a depth probe and guiding pin; and
all surgical and dental armamentarium required for any biological implant placement or removal.

11. The surgical instrument of claim 9, wherein the alloy material further comprises at least one of: about 0.01-10. wt.% ZrO_{2,} MgO, about 0.01-10. wt.% CaO, about 0.01-10. wt.% Ta₂O₅ about 0.01-10. wt.% Al₂O₃ about 0.01-47. wt.% Ag, about 0.01-50. wt.% Mg, and about 0.01-50. wt.% Zn.

12. The surgical instrument of claim 9, wherein the alloy material further comprises about 1–49 vol.% of a Ti₂Cu phase.

13. The surgical instrument of claim 11, wherein the alloy material further comprises one of: about 0.01-47. vol.% of a Ti₂Ag phase, about 0.01-50. vol.% of a Ti₂Mg phase or Ti-Mg Intermetallic, and about 0.01-50. vol.% of a Ti₂Zn phase.

14. An orthopedic implant comprising an alloy material, the alloy material comprising about 50–99.99 wt.% Ti, about 1–49 wt.% Cu, and about 0.01–10 wt.% SiO_{2.}

15. The orthopedic implant of claim 14, wherein the orthopedic implant is one of:
a jaw implant;
a spinal implant;
a hip implant;
a shoulder implant;
a fixation plate comprising a fixation screw;
a reconstruction plate comprising a reconstruction screw;
a pin;
a wire; and
a staple.

16. The orthopedic implant of claim 14, wherein the alloy material further comprises at least one of: about 0.01-10. wt.% ZrO_{2,} MgO, about 0.01-10. wt.% CaO, about 0.01-10. wt.% Ta₂O₅ about 0.01-10. wt.% Al₂O₃ about 0.01-47. wt.% Ag, about 0.01-50. wt.% Mg, and about 0.01-50. wt.% Zn.

17. The orthopedic implant of claim 14, wherein the alloy material further comprises about 1–49 vol.% of a Ti₂Cu phase.

18. The orthopedic implant of claim 16, wherein the alloy material further comprises one of: about 0.01-47. vol.% of a Ti₂Ag phase, about 0.01-50. vol.% of a Ti₂Mg phase or Ti-Mg Intermetallic, and about 0.01-50. vol.% of a Ti₂Zn phase.
